(19) United States
(12) Patent Application Publication
(10) Pub. No.: US 2023/0111803 A1
(43) Pub. Date: Apr. 13, 2023

(54) METHOD OF TREATING AND PREVENTING OCULAR DISEASE WITH HSV-2 DELTA GD

(71) Applicant: Albert Einstein College of Medicine, Bronx, NY (US)

(72) Inventor: Betsy C. Herold, Rowayton, CT (US)

(21) Appl. No.: 17/913,707

(22) PCT Filed: Apr. 7, 2021
(86) PCT No.: PCT/US2021/026158

§ 371 Date: Sep. 22, 2022

Related U.S. Application Data
(60) Provisional application No. 63/006,342, filed on Apr. 7, 2020.

(51) Int. Cl. A61K 39/245; A61P 27/02

(52) U.S. Cl. CPC A61K 39/245; A61P 27/02; A61K 2039/505

(57) ABSTRACT

Methods of treating or preventing ocular disease caused by herpes simplex virus-1 infection are provided, and comprise administering to a subject an effective amount of a herpes simplex virus-2 (HSV-2) having a deletion of an HSV-2 glycoprotein D-encoding gene in the genome to treat or prevent ocular disease in the subject, wherein the HSV-2 is phenotypically complemented with an HSV-1 glycoprotein D by propagating the HSV-2 in a complementing cell expressing the HSV-1 glycoprotein D.

### FEDERAL RESEARCH STATEMENT

This invention was made with government support under grant number T32 AI007501 awarded by the National Institutes of Health (NIH) and grant number R01 AI17321 awarded by the National Institute of Allergy and Infectious Diseases (NIAID). The government has certain rights in the invention

### BACKGROUND

Herpes simplex virus serotype 1 (HSV-1) is a major global health problem with an estimated 3.72 billion people infected worldwide. HSV-1 causes oral and genital mucocutaneous disease, sporadic encephalitis, and is the leading cause of infectious corneal blindness in the US and Europe. HSV-1 results in 300,000 diagnoses of ocular disease in the United States annually and 40,000 new cases of severe visual impairment globally. Most of these epidemiological studies have been limited to developed nations however, and thus may not accurately reflect the true global incidence. Ocular disease may occur in response to direct inoculation following primary exposure, or more commonly, following reactivation of virus that established latency in the trigeminal ganglia (TG) after oral infection. Primary or reactivating ocular infection may result in blepharitis, conjunctivitis, keratitis, retinitis or scleritis. Keratitis is the most prevalent and may progress to involve the deeper stromal structures resulting in herpes stromal keratitis (HSK), which can lead to blindness. HSK reflects both the cytolytic effects of viral replication as well as the immune and inflammatory response and is characterized by scarring, edema, neovascularization, and leukocyte infiltration.

The primary approach to HSV prevention has focused on genital disease and has centered on subunit protein vaccines compromised of the HSV-2 envelope glycoprotein D (gD-2) combined with different adjuvants. The subunit protein vaccines primarily elicit antibodies that neutralize both HSV-1 and HSV-2 in cell culture assays. These vaccines exhibit variable protection in preclinical models of vaginal, skin, and ocular disease with either serotype, but fail to provide significant protection against genital HSV-2 infection or disease, which was the primary study outcome, in clinical trials. Partial protection against genital HSV-1 disease was observed and correlated with neutralizing titers in an analysis of a small subset of participants. No clinical trials have been conducted to assess vaccine efficacy against HSV-1 ocular disease.

A genetically modified, single-cycle HSV-2 strain deleted in glycoprotein D (gD) has been developed to generate a single-cycle candidate HSV-2 vaccine strain designated ΔgD-2. The virus is grown on complementing cells that express HSV-1 gD (VD60 cells), to yield genotypically gD-null viruses that have incorporated gD-1 on their envelope, allowing for an initial cycle of infection. Vaccination with the complemented virus causes no disease in wild-type or immunodeficient mice because the viral progeny do not have gD on their envelope and thus are unable to infect new cells. The ΔgD-2 vaccine elicits high titer, polyantigenic IgG responses that provided protection in mice following lethal vaginal (female) or skin (female and male) inoculation with clinical isolates of HSV-1 or HSV-2. The vaccine also prevented the establishment of latency. Passive transfer studies demonstrated that the elicited antibodies (Abs) protected wild-type, but not Fc gamma receptor (FcγR) or neonatal Fc receptor (FcRn) knockout mice. The Abs had little complement-independent neutralizing activity, but activated murine FcγRIII and FcγRIV to mediate antibody dependent cell killing by cytolytic and phagocytic pathways.

However, there remains a need for a vaccine which can provide protection against ocular disease caused by HSV-1.

### SUMMARY

Disclosed herein is a method of treating or preventing ocular disease caused by herpes simplex virus-1 (HSV-1) infection comprising administering to a subject an effective amount of a herpes simplex virus-2 (HSV-2) having a deletion of an HSV-2 glycoprotein D-encoding gene in the genome to treat or prevent ocular disease in the subject, wherein the HSV-2 is phenotypically complemented with an HSV-1 glycoprotein D by propagating the HSV-2 in a complementing cell expressing the HSV-1 glycoprotein D.

Disclosed herein is a method of treating or preventing ocular infection by HSV-1 in a subject comprising administering to the subject an effective amount of a herpes simplex virus-2 (HSV-2) having a deletion of an HSV-2 glycoprotein D-encoding gene in the genome to treat or prevent ocular infection by HSV-1 in the subject, wherein the HSV-2 is phenotypically complemented with an HSV-1 glycoprotein D by propagating the HSV-2 in a complementing cell expressing the HSV-1 glycoprotein D.

Disclosed herein is a method of preventing or treating ocular disease caused by herpes simplex virus-1 (HSV-1) infection in a first subject comprising administering to the first subject an effective amount of a product from a second subject to prevent or treat the ocular disease in the first subject, wherein the second subject is immunized with a herpes simplex virus-2 (HSV-2) having a deletion of an HSV-2 glycoprotein D-encoding gene in the genome and wherein the HSV-2 is phenotypically complemented with a HSV-1 glycoprotein D by propagating the HSV-2 in a complementing cell expressing the HSV-1 glycoprotein D, and wherein the product comprises an antibody, an immune factor, or a combination thereof induced by the HSV-2.

Also disclosed herein is a method of preventing or treating ocular disease caused by herpes simplex virus-1 (HSV-1) infection in a first subject comprising administering to the first subject an effective amount of an antibody specific for an antigen of the HSV-1 to prevent or treat the ocular disease in the first subject, wherein the antibody is obtained by immunizing a second subject with a herpes simplex virus-2 (HSV-2) having a deletion of an HSV-2 glycoprotein D-encoding gene in the genome and wherein the HSV-2 is phenotypically complemented with an HSV-1 glycoprotein D by propagating the HSV-2 in a complementing cell expressing the HSV-1 glycoprotein D, and wherein the antibody elicited by immunizing the second subject with the HSV-2 comprises an Fc receptor-activating antibody.

The above described and other features are exemplified by the following figures and detailed description.

### BRIEF DESCRIPTION OF THE DRAWINGS

Those of skill in the art will understand that the drawings, described below, are for illustrative purposes only. The drawings are not intended to limit the scope of the present teachings in any way.

FIGS. 1A-D. ΔgD-2 vaccine protects against disease and latency in an ocular lethal HSV-1 challenge model. Female BALB/c mice received two doses of 5×10⁶ pfu ΔgD-2 (red), 5 micrograms (µg) recombinant glycoprotein D-2 (rgD-2) combined with alum and MPL (green) or an uninfected VD60 cell lysate as control (black) and were then challenged with 10⁵ pfu (FIG. 1A) (15 mice per group, 3 independent experiments) or 10⁶ pfu (FIG. 1B)(10 mice per group, 2 independent experiments) of HSV-1 (B3x1.1). Mice were scored for signs of disease: 0 - no disease; 1 - minimal eyelid swelling; 2 - moderate eyelid swelling, minimal ocular discharge, and/or minimal hair loss; 3 - severe eyelid swelling, moderate ocular discharge, and/or severe hair loss; 4 - eyes crusted shut; 5 - signs of neurologic disease (poor grooming, hunched back, signs of disequilibrium, paralysis, weight loss); 6-death. Mice were euthanized at a score of 5 and assigned a score of 6 the following day. Survival curves are shown to the right of disease scores. Viral spread to trigeminal ganglia was determined by quantitative PCR at time of demise because of disease or at day 28 post-challenge in survivors following infection with 10⁵ pfu (FIG. 1C) or 10⁶ pfu (FIG. 1D). Data is displayed as median copies per 10 ng of DNA. The disease scores were compared by two-way ANOVA with Sidak’s multiple comparison, survival by Gehan-Breslow-Wilcoxon test relative to controls and viral DNA by ANOVA with Tukey’s multiple comparison (*p<0.05, **p<0.01, ****p<0.0001).

FIGS. 2A-B. ΔgD-2 vaccination results in rapid clearance of virus and prevents spread to contralateral trigeminal ganglia. Female BALB/c mice received two doses of 5×10⁶ pfu ΔgD-2 (red), 5 µg rgD-2 combined with alum and MPL (green) or an uninfected VD60 cell lysate as control (black) and were then challenged with 10⁵ pfu HSV-1 (B3x1.1). (FIG. 2A) Eyes were swabbed on days 2, 3 and 5 and infectious virus quantified by performing a plaque assay on Vero cells. Results are presented as scatter plots with bar at median and compared at each time by ANOVA, *p< 0.05. The upper limit of detection for plaques per well is 150. (FIG. 2B) Contralateral trigeminal ganglia were excised at time of death and co-cultured with Vero cells to detect infectious or reactivating virus (n= 5 mice per group). Results are presented as percentage of positive cultures at each time point and compared using Gehan-Breslow-Wilcoxon test; ****p< 0.0001 for ΔgD-2 compared to both VD60 and adjuvanted rgD-2.

FIGS. 3A-B. Passive transfer of immune serum from mice vaccinated with ΔgD-2 protects against lethal ocular disease. Female BALB/c mice received two doses of 5×10⁶ pfu ΔgD-2 (red), 5 µg rgD-2 combined with alum and MPL (green) or an uninfected VD60 cell lysate (black) administered subcutaneously at three-week intervals. Mice were bled two weeks and four weeks after the second vaccine dose and the serum was pooled and total IgG quantified by ELISA. Naive mice were administered serum containing 750 µg IgG intraperitoneally one day prior and 4 days following corneal inoculation with 10⁵ pfu HSV-1 (B3x1.1). (FIG. 3A) The disease scores (left) were compared by two-way ANOVA with Sidak’s multiple comparison and survival (right) by Gehan-Breslow-Wilcoxon test, n=10 mice per group, **p< 0.01, ***p< 0.001 and****p< 0.0001. (FIG. 3B) Viral spread to trigeminal ganglia was determined by quantitative PCR at time of demise because of disease or at day 21 post-challenge in survivors and is presented as HSV DNA copies per 10 ng of DNA. N=9-10 mice/group in 2 independent experiments; ***p<0.001,****p<0.0001.

FIGS. 4A-F. Differences in passive protection reflect distinct antibody function. (FIG. 4A) Immune serum was assayed for total HSV-specific IgG by ELISA. Results are presented as optical densitometry (OD) units at indicated serum dilution with mean ± SEM (n=5 mice per group); **p< 0.01, linear regression. (FIG. 4B) Immune serum from mice with similar HSV-specific IgG titers were pooled and the new pools were retested in the HSV ELISA; ns (no significant difference in curves by linear regression). (FIG. 4C) Passive transfer studies were repeated with new pools of immune serum containing similar total HSV-specific IgG, n= 5 mice per group. Survival curves were compared by Gehan-Breslow-Wilcoxin, ****p< 0.0001 compared to VD60 or adjuvanted rgD-2. (FIG. 4D) Neutralization of viral infection was determined by plaque assay with indicated serum dilution and is shown as percent reduction in pfu relative to control serum. The horizontal line at 50% indicates the dilution of serum that inhibits 50% of viral plaques (neutralization titer, NT). The figure is representative of 3 mice. NT-1 (mean ± SEM) were determined based on n=5-6 mice and were 33±5.2 and 156±39.7 for ΔgD-2 and rgD-2, respectively; *** p< 0.001 unpaired t test with Welch’s correction area under curve. (FIG. 4E) ADCC was assayed using the murine FcγRIV activation assay with 1:5 dilution of immune serum; results are fold induction relative to controls and data shown as scatter plots, n=10 mice per group. ****p< 0.0001 ANOVA with Tukey’s multiple comparison (FIG. 4F) Clq binding of immune serum was assayed by ELISA at the indicated dilutions and results are shown as OD mean ± SEM, n=4 mice per group. *p< 0.0001 area under curve was compared by unpaired t test with Welch’s correction.

FIGS. 5A-E. Depletion of T cells in vaccinated mice prior to challenge results in reduction in protection for both vaccines. Mice received two doses (three weeks apart) of 5×10⁶ pfu ΔgD-2 (red), 5 µg rgD-2 combined with alum and MPL (green) or an uninfected VD60 cell lysate as control (black). Three weeks later, mice were treated with anti-CD4 and anti-CD8 monoclonal antibodies or an isotype control, which were administered intraperitoneally four and two days prior to challenge. (FIG. 5A) The percentage of CD4 and CD8 T cells in blood obtained one day prior to challenge was quantified by flow cytometry; results are presented as mean ± SEM (n=3 per group). Mice were challenged on the cornea (FIG. 5B) or intravaginally (FIG. 5C) and disease scores monitored (n=8-10 mice/group). (FIG. 5D) Mice were treated with anti-CD4 mAb or isotype control and challenged intraocularly (n=5 mice per group). Disease scores were compared between mice treated with depleting antibodies or isotype control antibody by two-way ANOVA with Sidak’s multiple comparisons test, *p<0.05, **p<0.01, ***p<0.001, ****p<0.0001 (FIG. 5E) Immune serum was assayed for total HSV-specific IgG by ELISA using serum obtained 5 days post-infection; differences between mice treated with depleting anti-CD4 or isotype control were compared by t-test.

### DETAILED DESCRIPTION

The inventors have discovered and demonstrated that immunization with a genetically modified, single-cycle, herpes simplex virus-2 (HSV-2) having a deletion of glycoprotein D in the genome (ΔgD-2), provides both active and passive protection against primary lethal corneal disease in a murine model. This was a surprising and unexpected result because the eye is a relatively immune privileged site and the ability of a vaccine to prevent ocular disease caused by HSV (e.g., HSV-1) differs from the ability of a vaccine to prevent vaginal or skin disease. The development of a vaccine to prevent ocular disease is also challenging because the eye is susceptible to immune mediated damage. Mechanisms understood to contribute to immune privilege within the eye include lack of direct lymphatic drainage, a blood-ocular barrier, as well as immunosuppressive and immunoregulatory responses. Additionally, the FcRn may preferentially transport immunoglobulin G (IgG) out of the eye into the systemic circulation, which could limit Ab-mediated protection. Further, because antibodies elicited by the ΔgD-2 vaccine function primarily by activating Fc receptors to mediate antibody-dependent cellular cytotoxicity (ADCC), the ability of ADCC to protect the immune privileged eye was previously unknown.

This disclosure shows that the genetically modified HSV (ΔgD-2), elicits antibodies that act primarily through their Fc component, and provides significantly greater protection following a two-dose vaccine regimen than an adjuvanted glycoprotein D subunit vaccine (rgD-2) that elicits neutralizing, but not Fc receptor activating or complement binding responses, in a primary lethal ocular murine model. Further, only immune serum from the mice vaccinated with the genetically modified HSV provided significant protection in passive transfer studies. The significantly greater passive protection afforded by persisted after controlling for total amount of HSV-specific IgG in the transferred serum. The antibodies elicited by the genetically modified HSV had significantly more C1q-binding and Fc gamma receptor-activation, a surrogate for ADCC function. These finding surprisingly suggest that ADCC is protective in the eye and that non-neutralizing antibodies provide greater protection against primary ocular HSV disease than neutralizing antibodies.

As used herein, “therapeutically effective amount” or “effective amount” refers to a quantity of a specific substance sufficient to achieve a desired effect in a subject being treated.

“Treat” or “treating,” means to administer a vaccine of the disclosure or a product of the disclosure to a subject or patient having one or more disease symptoms, or being suspected of having a disease, for which the vaccine or product has therapeutic activity or prophylactic activity. The vaccine or product can be administered in an amount effective to alleviate one or more disease symptoms in the treated subject, whether by inducing the regression of or inhibiting the progression of such symptom(s) by any clinically measurable degree. The terms further includes a postponement of development of the symptoms associated with a disorder and/or a reduction in the severity of the symptoms of such disorder. The terms further include ameliorating existing uncontrolled or unwanted symptoms, preventing additional symptoms, and ameliorating or preventing the underlying causes of such symptoms.

“Preventing” means administering an amount of a pharmaceutical formulation of the disclosure or a vaccine of the disclosure or a product of the disclosure which is sufficient to significantly reduce the likelihood of a disease from occurring in a subject who may be predisposed to the disease but who does not have it. In the context of viral infection “preventing” includes administering an amount of the vaccine or a product resulting from administration of the vaccine to a subject known to be at enhanced risk of viral infection.

Disclosed herein are methods of treating or preventing an ocular disease caused by a herpes simplex virus infection. In particular, the ocular disease is caused by a herpes simplex virus-1 (HSV-1) infection. Methods of treating or preventing ocular infection caused by a herpes simplex virus (e.g., HSV-1) are also disclosed. The methods comprise the administration of HSV-2, or a virion thereof, having a deletion of glycoprotein D-encoding gene in the genome, and phenotypically complemented with an HSV-1 glycoprotein D.

In an embodiment, a method of treating or preventing ocular disease caused by HSV-1 infection comprises administering to a subject an effective amount of a herpes simplex virus-2 (HSV-2) having a deletion of HSV-2 glycoprotein D-encoding gene in the genome of the HSV-2 to treat or prevent ocular disease in the subject, wherein the HSV-2 is phenotypically complemented with an HSV-1 glycoprotein D by propagating the HSV-2 in a complementing cell expressing HSV-1 glycoprotein D.

In an embodiment, a method of treating or preventing ocular infection by HSV-1 in a subject comprises administering to the subject an effective amount of a HSV-2 having a deletion of HSV-2 glycoprotein D-encoding gene in the genome of the HSV-2 to treat or prevent ocular disease in the subject, wherein the HSV-2 is phenotypically complemented with an HSV-1 glycoprotein D by propagating the HSV-2 in a complementing cell expressing the HSV-1 glycoprotein D.

The deletion of the HSV-2 glycoprotein D-encoding gene in the genome of the HSV-2 comprises a partial deletion of the HSV-2 glycoprotein D-encoding gene or a deletion of the entire HSV-2 glycoprotein D-encoding (gD) gene. The HSV-2 is phenotypically complemented with a herpes simplex virus-1 (HSV-1) glycoprotein D by propagating the HSV-2 in a complementing cell expressing the HSV-1 glycoprotein D. The HSV-2 having the deletion of the HSV-2 gD-encoding gene in the genome is a genetically modified (e.g., recombinant) HSV-2. The HSV-2 having a deletion of the HSV-2 glycoprotein D-encoding gene in the genome of the HSV-2, and which is phenotypically complemented with an HSV-1 glycoprotein D by propagating the HSV-2 in a complementing cell expressing the HSV-1 glycoprotein D, is referred to herein interchangeably as “HSV-2 ΔgD-2” or “ΔgD-2” or “genetically modified HSV-2.” The HSV-2 ΔgD-2 is a single-cycle virus.

Disclosed herein are methods of treating or preventing ocular disease in a subject comprising administering to a subject an effective amount of the HSV-2 ΔgD-2 to treat or prevent the ocular disease and/or to treat or prevent ocular infection. In an embodiment, the method comprises administering to a subject an effective amount of the HSV-2 ΔgD-2 to treat or prevent ocular infection by HSV-1 in the subject. The effective amount of the HSV-2 ΔgD-2 administered to the subject is an amount effective to elicit an immune response to HSV-1 and/or HSV-2 in the subject. In the methods disclosed herein, the elicited immune response effectively prevents or treats infection of a subject with HSV-1 and/or prevents or treats ocular disease caused by infection with HSV-1.

The immune response elicits antibodies, cellular immune responses, and/or other immune factors (e.g., complement) that minimize and/or prevent viral dissemination and/or viral infection in the eye of the subject. In particular, the immune response comprises the production of antibodies that activate Fc receptors to mediate an antibody-dependent cellular cytotoxicity (ADCC) response. The administration of an effective amount of the HSV-2 ΔgD-2 thus elicits the production of Fc receptor (FcR)-activating antibody (also referred to as antibody dependent cellular cytotoxicity (ADCC) antibody). The effective amount of HSV-2 ΔgD-2 is an amount of plaque forming units (pfu) of the HSV-2 ΔgD-2 which achieves the stated aim.

The subject is a subject in need of treatment or prevention of ocular disease caused by HSV-1. The subject can also be a subject in need of treatment or prevention of ocular infection by HSV-1. The subject is a mammalian subject. For example, the subject is a human subject. The HSV-2 ΔgD-2 can be formulated for administration to a human subject.

The ocular disease caused by HSV-1 comprises blepharitis, conjunctivitis, keratitis, retinitis, scleritis, or a combination thereof. Accordingly, the methods encompass treating or preventing blepharitis, conjunctivitis, keratitis, retinitis, scleritis, or a combination thereof caused by an HSV-1 infection.

The HSV-2 ΔgD-2 can be administered to a subject which is receiving or has received an anti-viral drug. The anti-viral drug is a small molecule anti-viral drug such as acyclovir, famcyclovir, penciclovir, valacyclovir, or a combination thereof. In an embodiment, the small molecule anti-viral drug is acyclovir.

In an embodiment, the subject is infected with HSV-1. In other embodiments, the subject is not yet infected with HSV-1. In an embodiment, the subject is immunocompromised. In an embodiment, the immunocompromised patient has cancer, has undergone a transplant, or is on an immunosuppressive medication. In an embodiment, the subject is pregnant. In an embodiment, the subject is a neonate.

The HSV-2 ΔgD-2 can be administered as a component of a pharmaceutical formulation. The pharmaceutical formulation can further include a pharmaceutically acceptable carrier, an immunological adjuvant, or a combination thereof. In an embodiment, the method of preventing or treating ocular disease caused by HSV-1 infection comprises administering an effective amount of a pharmaceutical formulation comprising the HSV-2 ΔgD-2 to a subject.

In an embodiment, the subject is receiving or has received an anti-viral therapy. The anti-viral therapy comprises administration of an anti-viral small molecule to the subject. The anti-viral small molecule is not particularly limited, and can be any anti-viral small molecule capable of treating or preventing a herpes virus infection in a subject. The anti-viral small molecule comprises famcyclovir, penciclovir, valacyclovir, or a combination thereof. In an embodiment, the anti-viral therapy comprises administration of the acyclovir to the subject.

Disclosed herein also is a method of preventing or treating ocular disease caused by HSV-1 infection in a first subject administering to the first subject an effective amount of a product from a second subject to prevent or treat the ocular disease in the first subject, wherein the second subject is immunized with HSV-2 ΔgD-2, and wherein the product comprises an antibody, an immune factor, or a combination thereof induced by the HSV-2 ΔgD-2. The product can further comprise the HSV-2 ΔgD-2. The product is administered to the first subject in an amount effective to elicit an immune response in the first subject against the HSV-1. In an embodiment, a method of preventing or treating ocular infection by HSV-1 also comprises administering to the first subject an effective amount of the product from a second subject to prevent or treat ocular infection by HSV-1 in the first subject.

The product is obtained by immunizing (vaccinating) the second subject with the HSV-2 ΔgD-2 in an amount effective to elicit an immune response to HSV-1 in the second subject. The product includes an antibody, an immune factor, or a combination thereof elicited by the immunization of the second subject with HSV-2 ΔgD-2. For example, the antibody, the immune factor, or the combination thereof present in the product and passively transferred to the first subject, is able to minimize and/or prevent HSV-1 dissemination and/or infection in the eye of the first subject.

The product can be obtained by immunizing the second subject with HSV-2 ΔgD-2, and after a predetermined time, removing a sample comprising the product from the second subject. The sample comprises, for example, blood, serum, plasma, breast milk, or a combination thereof. In an embodiment, the sample comprises serum of the second subject.

The product administered to the first subject can be removed from the second subject prior to its administration to the first subject. In an embodiment, administering the product comprises removing a sample comprising the product from the second subject and administering the sample to the first subject. The sample comprising the product can be administered to the first subject without further processing, and/or the sample can be processed (e.g., diluted, concentrated) to optimize the amount of product prior to its administration to the first subject. The product can also be isolated from the sample using methods known in the art. The product can be administered as a component of a pharmaceutical formulation.

The product administered to the first subject can be directly transferred from the second subject to the first subject. In an aspect, the administering comprises direct transfer of the sample comprising the product from the second subject to the first subject. For example, the administering can comprise passive transfer of a sample comprising the product from a mother to a fetus and/or a neonate.

The first subject and the second subject are each a mammalian subject. In an embodiment, the first subject and the second subject are each a human subject.

In the methods disclosed herein, the product comprises an antibody elicited by immunization of the second subject with the HSV-2 ΔgD-2. The antibody elicited by immunization activates Fc receptors to mediate an antibody-dependent cellular cytotoxicity (ADCC) response. The antibody thus comprises a Fc receptor (FcR)-activating antibody (ADCC antibody). In an embodiment, the product comprises anti-HSV-1 IgG. The antibody can comprise a polyclonal antibody, a monoclonal antibody, or a combination thereof. The antibody can be present in the sample from the second subject.

Also disclosed herein is a method of preventing or treating ocular disease caused by herpes simplex virus-1 (HSV-1) infection in a first subject comprising administering to the first subject an effective amount of an antibody specific for an antigen of the HSV-1 to prevent or treat the ocular disease in the first subject, wherein the antibody is obtained by immunizing a second subject with herpes simplex virus-2 (HSV-2) having a deletion of an HSV-2 glycoprotein D-encoding gene in the genome of the HSV-2 and wherein the HSV-2 is phenotypically complemented with a herpes simplex virus-1 (HSV-1) glycoprotein D by propagating the HSV-2 in a complementing cell expressing the HSV-1 glycoprotein D, and wherein the antibody elicited by immunization of the second subject with the HSV-2 comprises a Fc receptor-activating antibody.

As used herein, “antibody” refers to an intact (whole) immunoglobulin (i.e. with complete Fc and Fv regions), including recombinantly produced forms, and includes any form of antibody that exhibits the desired biological activity. Thus, the term is used in the broadest sense and specifically covers, but is not limited to, monoclonal antibodies (including full length monoclonal antibodies), polyclonal antibodies, multispecific antibodies (e.g., bispecific antibodies), humanized antibodies, fully human antibodies, biparatopic antibodies, and chimeric antibodies. A “parental antibody” is an antibody obtained by exposure of an immune system to an antigen prior to modification of the antibodies for an intended use, such as humanization of an antibody for use as a human therapeutic antibody. The term “Fc region” is a C-terminal region of an immunoglobulin heavy chain that contains at least a portion of the constant region. The “Fv region” comprises the variable regions from both the heavy and light chains, but lacks the constant regions. As used herein, the term “isolated antibody” refers to an antibody that by virtue of its origin or source of derivation has at least one (e.g., one, two, three, or four) of the following: (1) is not associated with naturally associated components that accompany it in its native state, (2) is free of other proteins from the same species, (3) is expressed by a cell from a different species, or (4) does not occur in nature absent the hand of man.

Intact antibodies are glycoproteins comprising at least two heavy (H) chains and two light (L) chains interconnected by disulfide bonds. Each heavy chain is comprised of a heavy chain variable region (V_{H}) and a heavy chain constant region. The heavy chain constant region is comprised of three domains, CH1, CH2 and CH3. Each light chain is comprised of a light chain variable region (V_{L}) and a light chain constant region. The light chain constant region is comprised of one domain, CL. The V_{H} and V_{L} regions are further subdivided into regions of hypervariability, termed complementarity determining regions (CDR), interspersed with regions that are more conserved, termed framework regions (FR). Each VH and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain or binding portion that interacts with an antigen.

The antibody can be a fully human antibody. As used herein, a “fully human antibody” refers to an antibody that comprises only human immunoglobulin amino acid sequences or variant sequences thereof comprising mutations introduced recombinantly to provide a fully human antibody with modified function or efficacy compared to the antibody lacking said mutations.

The antibody can be a parental human antibody or a humanized antibody. As used herein, a “humanized antibody” refers to forms of antibodies that contain sequences from both human and non-human (e.g., murine, rat) antibodies. Humanized forms of non-human (e.g., murine) antibodies are chimeric antibodies that contain minimal sequence derived from non-human immunoglobulin. In an embodiment, a humanized antibody is an antibody having the sequence of a human immunoglobulin (recipient antibody) but the residues from a murine hypervariable region (HVR) (or complementarity determining region, CDR). In an embodiment, framework (FR) residues of the murine mAb are replaced with corresponding human immunoglobulin variable domain framework (FR) residues. These humanized antibodies may be modified further to refine antibody performance. The humanized antibodies may comprise residues that are not found in the recipient antibody or in the donor antibody. Alternatively, the humanized antibodies do not comprise residues that are not found in the recipient antibody or in the donor antibody. In general, a humanized antibody will comprise substantially all of at least one, and typically two, variable domains, in which all, or substantially all, of the hypervariable loops correspond to those of a non-human immunoglobulin, and all, or substantially all, of the FRs are those of a human immunoglobulin sequence. The humanized antibody optionally will also comprise at least a portion of an immunoglobulin constant region (Fc), typically that of a human immunoglobulin. (See, e.g., Jones et al., Nature 321:522-525 (1986); Riechmann et al., Nature 332:323-329 (1988); Presta, Curr. Op. Struct. Biol. 2:593-596 (1992); Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409, the contents of each of which are hereby incorporated by reference in their entirety.) In an embodiment the humanized antibodies comprise residues that are not found in the recipient antibody or in the donor antibody, and the Fc regions of the humanized antibodies are modified as described in WO 99/58572, the content of which is hereby incorporated by reference in its entirety. Techniques to humanize a monoclonal antibody are known and are described in, for example, U.S. Pat. Nos. 4,816,567; 5,807,715; 5,866,692; 6,331,415; 5,530,101; 5,693,761; 5,693,762; 5,585,089; and 6,180,370, the content of each of which is hereby incorporated by reference in its entirety.

The antibody can comprise an antigen-binding fragment. As used herein, the term “antigen-binding fragment” refers to any portion of an antibody, or portions of an antibody linked together, which is less than the whole antibody, but which retains the ability to specifically bind to an antigen. The antigen-binding fragment competes with the intact antibody of which it is a fragment for specific binding. In this case, the antigen is an HSV-1 antigen. The antigen may also be an HSV-2 antigen. An “antibody” or a “fragment” thereof can comprise an immunoglobulin of any class, e.g., IgG, IgM, IgA, IgD and IgE. In an embodiment, the class is IgG and the isotype is IgG2c. Examples encompassed by “antigen-binding fragment” include a Fab fragment (a monovalent fragment consisting of the V_{L} V_{H}, C_{L}, and C_{Hl} domains), a F(ab′)₂ fragment (a bivalent fragment comprising two Fab fragments linked by a disulfide bridge at the hinge region), a Fab’ fragment (monovalent fragment produced by reduction of F(abʹ)₂ fragment, and which have a free sulfhydryl group), a fragment consisting of the V_{L} and V_{H} domains of a single arm of an antibody, a Fv fragment (a fragment consisting of the V_{L} and V_{H} domains of a single arm of an antibody), a single chain fragment (scFv, a variable domain light chain (VL) and a variable domain heavy chain (VH) linked via a peptide linker), a dAb fragment (consists of a V_{H} domain); an isolated complementarity determining region (CDR), a nanobody (a heavy chain variable region containing a single variable domain and two constant domains), mutants thereof, naturally occurring variants, fusion proteins comprising an antibody portion with an antigen recognition site of the required specificity, humanized antibodies, chimeric antibodies, and any other modified configuration of the immunoglobulin molecule that comprises an antigen recognition site of the required specificity. The antigen-binding fragment can be a polypeptide that contains at least a portion of an antibody that is sufficient to confer HSV-1 antigen -specific binding on the polypeptide.

The antibody can be produced recombinantly. For example, an antibody expressed using a recombinant expression vector transfected into a host cell, an antibody isolated from a recombinant combinatorial human antibody library, an antibody isolated from an animal (e.g., a mouse) that is transgenic for human immunoglobulin genes, or a combination thereof.

The term “monoclonal antibody” or “mAb” refers to an antibody member of a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In contrast to polyclonal antibody preparations, which include different antibodies directed against different antigenic determinants (epitopes), a monoclonal antibody is directed against a single determinant on an antigen. In addition to their specificity, monoclonal antibody preparations are advantageous in that they are typically uncontaminated by other immunoglobulins. Thus an identified monoclonal antibody can be produced by non-hybridoma techniques, e.g. by appropriate recombinant means, once the sequence thereof is identified.

A pharmaceutical formulation comprising an effective amount of the antibody specific for an antigen of the HSV-1 can be administered to the first subject to prevent or treat the ocular disease in the first subject. A pharmaceutical formulation comprising an effective amount of the antibody specific for an antigen of the HSV-1 can also be administered to the first subject to prevent or treat ocular infection by HSV-1.

The pharmaceutical formulations disclosed herein can include a pharmaceutically acceptable carrier. As used herein, the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals. The term “pharmaceutically acceptable carrier” refers to an excipient, diluent, preservative, solubilizer, emulsifier, adjuvant (also referred to as immunological adjuvant), and/or vehicle with which the present antibody or fragment is administered. Examples of pharmaceutically acceptable carriers include, but are not limited to, phosphate buffered saline solution, sterile water (including water for injection USP), emulsions such as oil/water emulsion, and various types of wetting agents. Preferred diluents for aerosol or parenteral administration are phosphate buffered saline or normal (0.9%) saline, for example 0.9% sodium chloride solution, USP. Compositions comprising such carriers are formulated by well-known conventional methods (see, for example, Remington’s Pharmaceutical Sciences, 18th edition, A. Gennaro, ed., Mack Publishing Co., Easton, Pa., 1990; and Remington, The Science and Practice of Pharmacy 20th Ed. Mack Publishing, 2000, the content of each of which is hereby incorporated in its entirety). In non-limiting examples, the can comprise one or more of dibasic sodium phosphate, potassium chloride, monobasic potassium phosphate, polysorbate 80 (e.g. 2-[2-[3,5-bis(2-hydroxyethoxy)oxolan-2-yl]-2-(2-hydroxyethoxyjethoxy]ethyl (E)-octadec-9-enoate), disodium edetate dehydrate, sucrose, monobasic sodium phosphate monohydrate, and dibasic sodium phosphate dihydrate. Except insofar as any conventional media or agent is incompatible with the HSV-2 ΔgD-2 or product, such use in the pharmaceutical formulation is contemplated.

The pharmaceutical formulations are formulated to be suitable for the intended route of administration to a subject. For example, the pharmaceutical formulation may be formulated to be suitable for intravenous, oral, intraperitoneal, intranasal, intratracheal, subcutaneous, intramuscular, topical, intradermal, transdermal or pulmonary administration. In an embodiment, the HSV-2 ΔgD-2 or pharmaceutical formulation comprising the HSV-2 ΔgD-2 can be formulated so that it is suitable for administration to a human subject. In an embodiment, the pharmaceutical formulation comprising the HSV-2 ΔgD-2 or the product from the second subject is formulated so that it is suitable for subcutaneous, oral, intramuscular, intra-nasal, intravaginal, or mucosal administration to a human subject.

The pharmaceutical formulation can comprise an immunological adjuvant. In an embodiment, a pharmaceutical formulation comprising the HSV-2 ΔgD-2 comprises an immunological adjuvant. The term “adjuvant” refers to a compound that when administered in as part of a pharmaceutical composition described herein augments, enhances and/or boosts the immune response to HSV-2 ΔgD-2, but when the compound is administered alone does not generate an immune response to the HSV-2 ΔgD-2. Non-limiting examples of adjuvants include alum, aluminum hydroxide, aluminum phosphate, calcium phosphate hydroxide, squalene, Quil A, MPIL, Freund’s adjuvant, oil in water emulsions (such as squalene or peanut oil), CpG. Adjuvants can be used with or without other specific immunostimulating agents such as MPL or 3-DMP, QS21, polymeric or monomeric amino acids such as polyglutamic acid or polylysine, or other immunopotentiating agents.

Pharmaceutical formulations disclosed herein can comprise a stabilizer to prevent loss of activity or structural integrity of the HSV-2 ΔgD-2, the product, and/or the antibody due to the effects of denaturation, oxidation or aggregation over a period of time during storage and transportation prior to use. The pharmaceutical formulation can comprise one or more of any combination of salts, surfactants, pH and tonicity agents such as sugars can contribute to overcoming aggregation problems. Where a pharmaceutical formulation of the present invention is formulated for injection, it is desirable to have a pH value in an approximately neutral pH range, and it is also advantageous to minimize surfactant levels to avoid bubbles in the formulation which are detrimental for injection into a subject. The pharmaceutical formulation can be in liquid form and can stably support high concentrations of bioactive antibody in solution. In an embodiment, the pharmaceutical formulation is suitable for intravenous, oral, intramuscular, intraperitoneal, intradermal, and/or subcutaneous injection. In an embodiment, the pharmaceutical formulation is in liquid form and has a minimized risk of bubble formation and anaphylactoid side effects. The pharmaceutical formulation can have a pH of 6.8 to 7.4. The pharmaceutical formulation can be isotonic.

In an embodiment the pharmaceutical formulation comprising the product, antibody, antigen-binding fragment, and/or immune factor described herein is substantially pure with regard to the antibody, or antigen-binding fragment thereof. A composition or pharmaceutical composition comprising the antibody, or antigen-binding fragment thereof, described herein is “substantially pure” with regard to the antibody or fragment when at least 60% to 75% of a sample of the composition or pharmaceutical composition exhibits a single species of the antibody, or antigen-binding fragment. A substantially pure composition or pharmaceutical composition comprising the antibody, or antigen-binding fragment thereof, described herein can comprise, in the portion thereof which is the antibody, or antigen-binding fragment, 60%, 70%, 80% or 90% of the antibody, or antigen-binding fragment, of the single species, more usually about 95%, and preferably over 99%. Purity or homogeneity may be tested by a number of means well known in the art, such as polyacrylamide gel electrophoresis or HPLC.

The HSV-2 ΔgD-2, product, antibody, and/or pharmaceutical formulation described herein can also be lyophilized and/or freeze dried and subsequently reconstituted for use, or provided in any suitable form including, but not limited to, injectable solutions or inhalable solutions, gel forms and tablet forms.

The effective amount of HSV-2 ΔgD-2 is an amount of plaque forming units (pfu) of the HSV-2 ΔgD-2 which achieves the stated aim. In particular, an effective amount is an amount of pfu of the HSV-2 ΔgD-2 which elicits an immune response in a subject sufficient to prevent or treat ocular disease caused by HSV-1 infection and/or prevent or treat ocular infection by HSV-1. The effective amount will generally be in a range of 10² to 10⁹ pfu, or 10³ to 10⁹ pfu, or 10⁴ to 10⁴ pfu, or 10⁴ to 10⁸ pfu. The HSV-2 ΔgD-2 can be administered as a single dose, or as a plurality of doses separated by a defined period of time. For example, a first dose of HSV-2 ΔgD-2 can be followed by a subsequent second dose a period to 2 to6 weeks, or 2 to 4 weeks after the first dose.

It is understood that aspects and embodiments of the invention described herein include “consisting” and/or “consisting essentially of” aspects and embodiments.

The terms “a” and “an” do not denote a limitation of quantity, but rather denote the presence of at least one of the referenced item. The term “or” means “and/or”. As used herein, the term “and/or” includes any and all combinations of one or more of the associated listed items. The open-ended transitional phrase “comprising” encompasses the intermediate transitional phrase “consisting essentially of” and the close-ended phrase “consisting of.” Claims reciting one of these three transitional phrases, or with an alternate transitional phrase such as “containing” or “including” can be written with any other transitional phrase unless clearly precluded by the context or art.

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in any suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

This disclosure is further illustrated by the following the Experimental Details, which are non-limiting.

### EXPERIMENTAL DETAILS

Female BALB/c mice were purchased from the Jackson Laboratory (JAX, Bar Harbor, ME). Vero (African Green Monkey Kidney, ATCC CCL-81) and VD60 cells were grown in DMEM (Invitrogen, Carlsbad, CA) supplemented with 10% FBS (Hyclone, Logan, UT) and 1% penicillin-streptomycin (Invitrogen). ΔgD-2 was propagated in VD60 cells and viral titers (plaque forming units (pfu)/ml)) were quantified on complementing VD60 and non-complementing Vero cells. HSV-1 strain B3x1.1 (hereinafter referred to as B3x1.1), a clinical isolate of HSV-1, was originally obtained from the Montefiore Clinical Virology Lab and propagated on Vero cells. Recombinant HSV-2 gD protein (rgD-2) was synthesized by the Einstein Protein Core Facility.

### Immunization and Challenge Protocol

Four to six-week-old female mice were immunized subcutaneously (sc) with 5 × 10⁶ plaque pfu of ΔgD-2 (based on the titer on VD60 cells; note no plaques were detected on Vero cells), 5 µg of rgD-2 combined with 150 µg alum (Imject Alum, Pierce Biotechnology, Rockland, IL) and 12.5 µg monophosphoryl lipid A (MPL) (Invivogen, San Diego, CA) (hereinafter referred to as “rgD-2/Alum-MPL”), or an uninfected VD60 cell lysate as a control. Two doses of vaccine were administered at three week intervals, and three weeks after the second dose, mice were challenged on the right eye with 10⁵ or 10⁶ pfu of Bx31.1 diluted in 5 µL of phosphate buffered saline (PBS) after scarifying the cornea with a 24-guage needle. Mice were monitored daily for signs of ocular disease and scored as follows: (0) no disease; (1) minimal eyelid swelling; (2) moderate eyelid swelling, minimal ocular discharge, or minimal hair loss; (3) severe eyelid swelling, moderate ocular discharge, or severe hair loss; (4) eyes crusted shut; (5) signs of neurologic disease (poor grooming, hunched back, disequilibrium, paralysis, weight loss); (6) death. Mice were euthanized at a score of 5 and assigned a score of 6 the following day. In select experiments, vaginal challenges were conducted for comparison using previously described methods (12, 18). Vaccinated mice were treated with 2.5 mg of medroxyprogesterone administered sc 5 days prior to intravaginal challenge with 5×10⁶ pfu of Bx31.1 diluted in 20 µL of PBS. Mice were monitored for two weeks and scored as follows: (0) no disease; (1) mild erythema or edema; (2) small lesion, moderate erythema, edema or hair loss at inoculum site; (3) large lesion, multiple lesions, or hair loss and/or mild paresis, urinary or fecal retention; (4) hind limb paralysis or severe urinary or fecal retention; (5) death. Mice with a score of 4 were euthanized and assigned a score of 5 the following day.

### Passive Transfer Studies

Serum was collected ~2 weeks post-second vaccine dose, randomly pooled and then assayed for total IgG content by ELISA (Catalog No.: 88-50400-88; Invitrogen, Carlsbad, CA). In select experiments, serum was pooled based on HSV-specific IgG titers, which were quantified using ELISA against HSV-infected cells as described below. Immune serum containing 750 µg of total IgG (final volume 250-300 µl) was administered intraperitoneally to naive (not previously immunized) mice 24 hours (h) before and four days after corneal challenge with 10⁵ pfu of B3x1.1.

### Quantitation of HSV DNA in Neuronal Tissue

At the time of euthanasia or when mice succumbed to disease, trigeminal or sacral dorsal root ganglia were harvested and DNA isolated using the Qiagen Blood and Tissue DNA isolation kit (Qiagen). Previously described primers and probes targeting the HSV polymerase gene were used to quantify viral DNA by quantitative polymerase chain reaction (qPCR) using 10 ng DNA per sample (Burn et al, 2017, The Journal of Infectious Diseases doi:10.1093/infdis/jix628). Mouse β-actin primers and probes were included as a loading control (Applied Biosystems, Foster City, CA), and qPCR was run in an Applied Biosystems QuantStudio 7 Flex. Samples with fewer than 4 copies detected were considered negative.

### Detection of Infectious Virus by Plaque Assay

On days 2, 3, 5, and 6 post-challenge, infected eyes were swabbed with sterile cotton tipped applicators pre-moistened with DMEM. The swabs were placed into 1 mL of DMEM in Eppendorf tubes and vortexed for 15 seconds to elute virus before discarding the swab. Samples were frozen at -80° C. until plaque assays on Vero cells were performed. Plaque assays were conducted by inoculating Vero cells grown on 24-well plates in duplicate with 250 µl of sample. Plaques were counted after 48 h.

To assess whether infectious virus had spread from inoculated eye to the contralateral side, contralateral TG were extracted at time of death or at day 28 post-challenge, minced and plating onto Vero cell monolayers grown on 6-well plates. Cells were monitored microscopically daily for plaque formation as evidence of viral reactivation.

### Antibody Assays

Total HSV-1-binding IgG titers were quantified by ELISA using HSV-1 infected Vero cells as the antigen. Vero cells were infected with B3x1.1 at a multiplicity of infection (MOI) of 0.1 pfu/cell and after 24 h incubation, the cells were harvested by scraping and sonicated for 30 seconds. Uninfected Vero cell lysates were prepared in parallel as controls. The protein concentration was determined by Pierce™ BCA Protein Assay (ThermoFisher Scientific, Waltham, MA) and 10 µg of infected or uninfected lysate were added to individual wells of a 96-well MaxiSorp ELISA plate (Nunc, NY). Plates were incubated overnight at 4° C. The cells were then further permeabilized with 0.1% Triton X-100 in PBS and fixed with 1% formaldehyde. Serially-diluted individual serum samples (10-fold dilutions from 1:1000-1:1,000,000) were added in duplicate to wells and allowed to bind overnight at 4° C. Wells were washed 5 times with 0.05% Tween 20 buffer, incubated with goat anti-mouse IgG biotin-labeled secondary antibody (Catalog #553999; BD Pharmingen, San Jose, CA) for an additional 2 h at room temperature (RT), washed again, incubated with horse radish peroxidase-conjugated streptavidin (BD Pharmingen, San Jose, CA) for 30 min at RT and then developed with tetramethylbenzidine (TMB) substrate (BD OptEIA) for 5 min. The reaction was stopped with 2 normal (N) H₂SO₄ and absorbance read at 450 nm on a SpectraMax (M5 series) ELISA plate reader. The final absorbance was determined by subtracting values obtained for uninfected cell lysates from values obtained with infected cell lysates.

Neutralization assays were conducted as previously described (Petro et al, 2015, Elife 4). Serial 4-fold dilutions of heat-inactivated serum were incubated with B3x1.1 (100 pfu/well) for 1 hour at 37° C. and then the mixture was added to a Vero cell monolayer for 1 hour at 37° C. Cells were fixed with methanol and stained with Giemsa after a 48-h incubation. Plaques were counted and the neutralization titer was defined as the highest dilution to result in a 50% reduction in plaque numbers.

ADCC was determined using the mFcγRIV ADCC Reporter Bioassay (Promega, Madison, WI). Vero cells were infected with HSV-1 B3x1.1 at a MOI of 0.1 pfu/cell for 12 hours as targets for the assay, transferred to white, flat-bottomed 96-well plates and incubated with heat-inactivated serum from immunized mice (1:5 dilution in DMEM) for 15 minutes at room temperature. FcγRIV-expressing reporter cells were added for 6 hours at 37° C., 5% CO₂ and FcγRIV activation was detected by the addition of luciferin substrate. Plates were read in a SpectraMax M5e (Molecular Devices). Fold induction was calculated relative to luciferase activity in the absence of serum.

Clq binding was assessed by ELISA. 96-well ELISA plates were coated with B3x1.1-infected or uninfected (control) Vero cell lysates as targets as for the ADCC assays. Wells were blocked for 1 hour at RT with 5% dry skim milk in PBS with 0.1% Igepal CA-630 (Sigma-Aldrich, Germany) and then washed four times. Serial four-fold dilutions of immune serum from individual mice were added to each well for an additional 2 h before washing. Murine Clq (Complement Technology, Tyler, TX) was added at 2 ug/mL and incubated for 2 hours at RT before washing. Bound C1q was quantified by adding rat anti-mouse Clq-biotin (0.5 µg/ml) (Cedarlane, Canada) for 1 hour at RT followed by incubation with HRP-conjugated streptavidin (BD Pharmingen, San Jose, CA).

### T Cell-Depletion Post-Active Immunization

Vaccinated mice were treated with 0.15 mg of anti-CD4 (clone GK1.5) alone or in combination with anti-CD8 (clone 2.43) monoclonal antibodies (mAbs) or an equivalent quantity of anti-rat keyhole limpet hemocyanin mAb as an isotype control administered intraperitoneally 4 days and 2 days prior to vaginal or ocular challenge. Depletion was assessed by flow cytometry. Tail blood was collected into Alsever’s solution, centrifuged at 1500 rpm at 4° C. for 5 min. Ammonium-chloride-potassium lysing buffer (ThermoFisher Scientific, Waltham, MA) was added to the pellet to lyse RBCs and samples were centrifuged at 1500 rpm, 4° C. for 5 min. Cell viability was assessed using the eFluor 450 fixable viability dye. Samples were incubated with anti-CD16/CD32 for 20 min at 4° C. for FcR receptor blocking, stained with a cocktail of mAbs comprised of anti-CD45 (FITC), anti-CD3 (PE-Cy7), anti-CD4 (PerCP-Cy5.5), and anti-CD8 (APC-H7 in FACS buffer (PBS supplemented with 2% (v/v) FBS) at 4° C. for 30 min. Samples were washed 3 times and then fixed with 4% (v/v) paraformaldehyde (Electron Microscopy Science) for 20 min at RT. Samples were analyzed with LSRII flow cytometer (BD) and FlowJo software. All antibodies were purchased from eBioscience (ThermoFisher Scientific, Waltham, MA).

### Statistical Analysis

Analyses were performed using GraphPad Prism version 8.0 software (GraphPad Software Inc. San Diego, CA). A P value of 0.05 was considered statistically significant. Survival curves were compared using the Gehan-Breslow-Wilcoxon test and other results were compared using ANOVA with multiple testing as indicated.

### Example 1: Immunization with ΔgD-2 Provides Greater Protection Against Lethal Ocular HSV-1 Infection

A stringent and lethal ocular disease model was developed in which the corneas of Balb/c mice were infected with different doses of the clinical isolate HSV-1 strain B3x1.1 (hereinafter referred to as B3x1.1), which has been shown to cause lethal disease following skin and vaginal challenge in adult mice and following intranasal infection of pups (Burn et al, 2017, J Infect Dis., doi: 10.1093/infdis/jix628; Petro et al, 2016, JCI Insight 1; Kao et al, 2019, J Infect Dis., doi: 10.1093/infdis/jiz521). B3x1.1 infection of the cornea resulted in consistent lethality at doses of 10⁵ pfu or greater, and doses of 10⁵ or 10⁶ pfu per animal was used for subsequent vaccine studies.

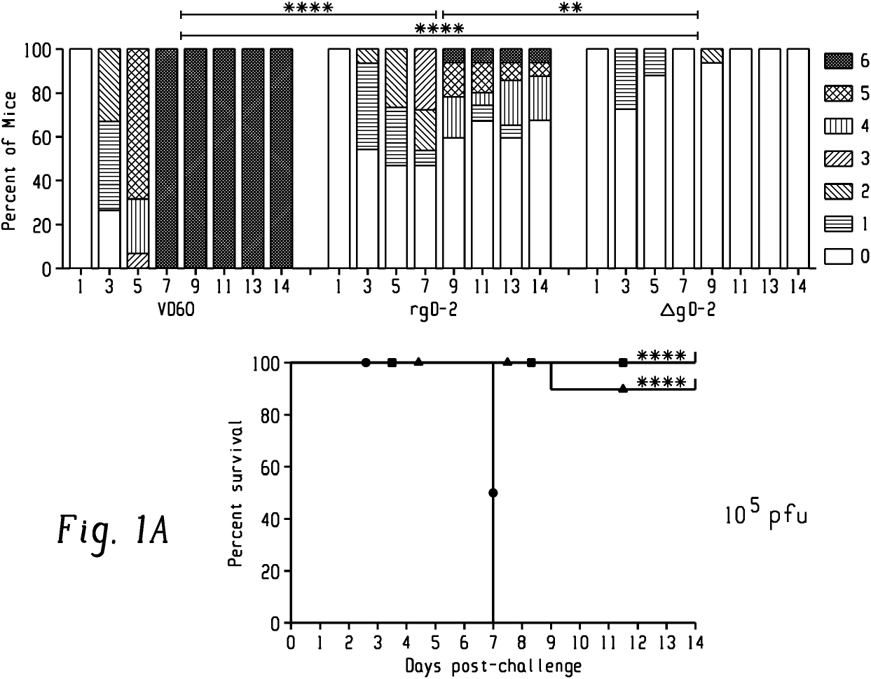

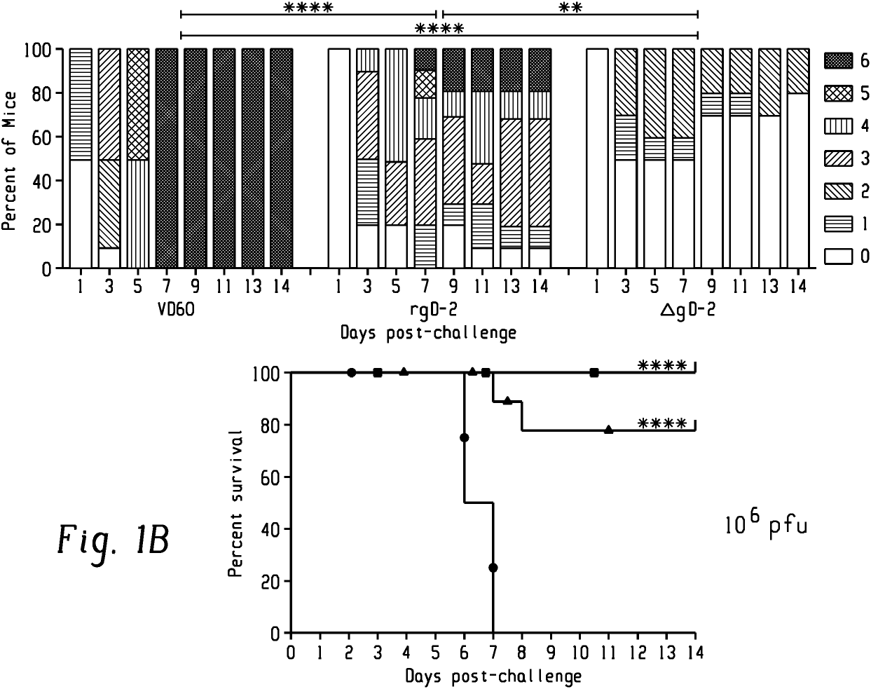

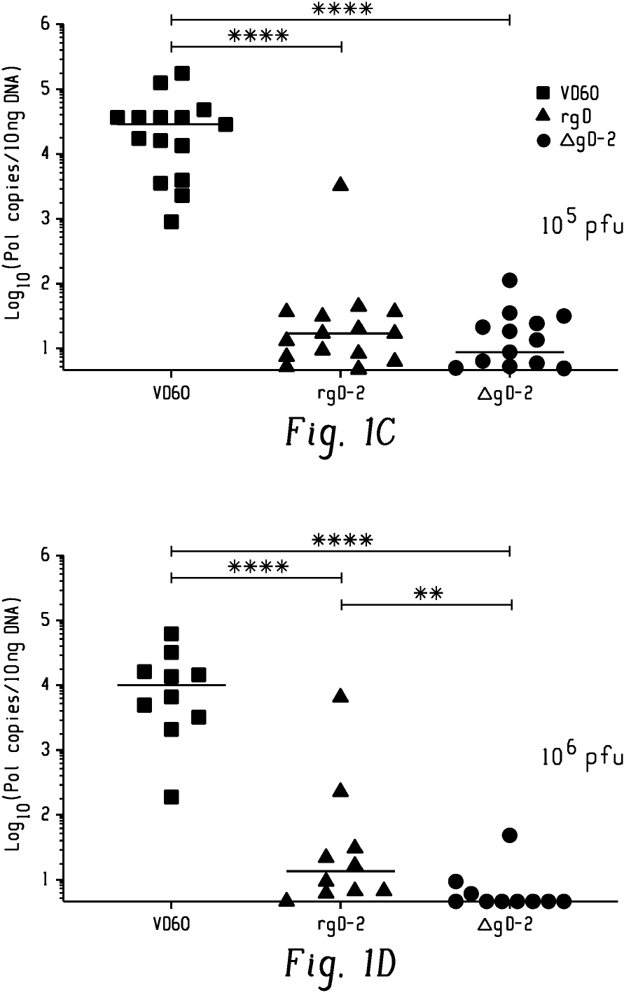

Female mice received two doses of ΔgD-2, two doses of recombinant glycoprotein D formulated with alum and MPL (rgD-2/Alum-MPL), which is similar to the vaccine used in the Herpevac Trial (7), or two doses of an uninfected VD60 cell lysate as a control prior to corneal challenge with 10⁵ (FIG. 1A) or 10⁶ (FIG. 1B) pfu of B3x1.1. Mice vaccinated with either ΔgD-2 or rgD-2/Alum-MPL exhibited significantly lower disease scores compared to VD60-vaccinated controls (p< 0.001). The controls developed progressive ocular and neurologic disease and succumbed by day 7 post-infection (pi). However, there were significant differences between the two vaccines. In particular, disease scores were significantly lower in ΔgD-2 vaccinated mice compared to rgD-2/Alum-MPL vaccinated mice (p <0.01), and this difference was magnified when the mice were challenged with 10⁶ pfu of B3x1.1 All of the mice infected with the higher dose of virus exhibited signs of disease as early as Day 3, but the ΔgD-2 vaccinated mice recovered quickly with no mortality. In contrast, 2/10 mice vaccinated with rgD-2/Alum-MPL succumbed and most exhibited persistent signs of disease (Scores >2) throughout the two-week monitoring period. Both vaccines provided significant protection against latency after challenge with 10⁵ or 10⁶ pfu, as evidenced by the quantity of HSV DNA detected in excised ipsilateral TG (FIGS. 1C and D, respectively).

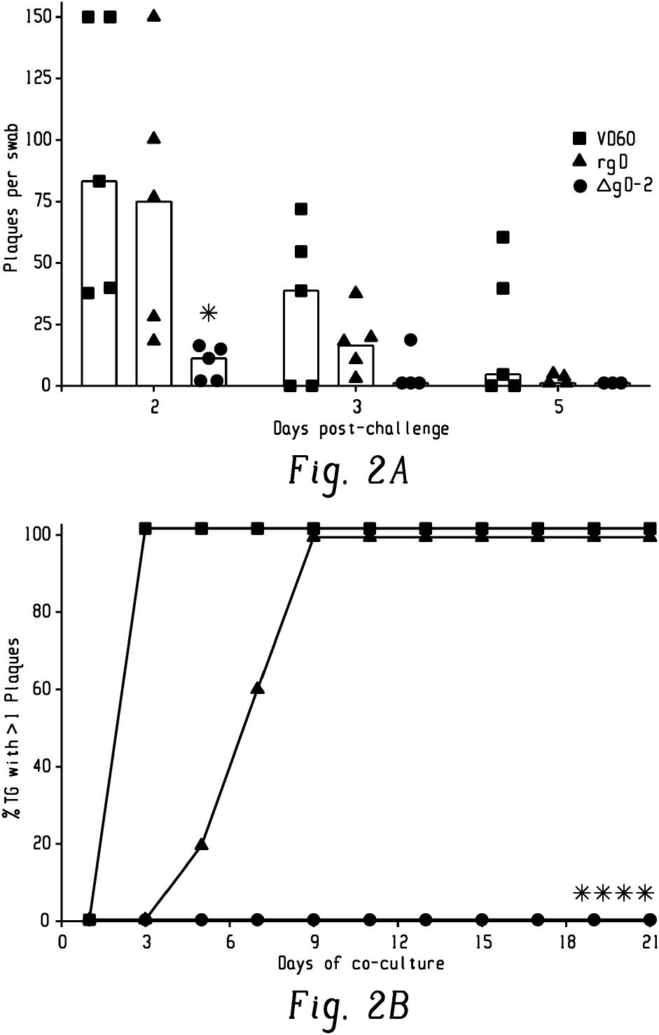

To assess how quickly virus was cleared from the site of inoculation, the eyes were swabbed, and infectious virus quantified by plaque assay (n=3 mice per group). Significantly less virus was detected in ΔgD-2 vaccinated compared to control mice 2 days post-inoculation (p=0.02) and declined more rapidly over the next several days although the differences in clearance did not reach statistical significance (FIG. 2A). To assess the ability of the vaccines to prevent viral spread, replicating or latent virus was quantified in the contralateral TG by co-culturing minced TG tissue with Vero cells (n=5 mice per group). No infectious virus was detected in TG harvested from ΔgD-2 vaccinated mice even after 21 d of co-culture, whereas virus was detected in all of the TG harvested from rgD-2/Alum-MPL vaccinated mice by Day 9 and within 48-72 h in all of the TG from control vaccinated mice (FIG. 2B, p<0.0001 ΔgD-2 versus rgD-2 or VD60 control). Virus detected within 2-3 days presumably reflects lytic virus rather than latent virus.

### Example 2: Transfer of Immune Serum from ΔgD-2, but Not rgD-2/Alum-MPL Vaccinated Mice, Protects Against Subsequent Ocular Challenge

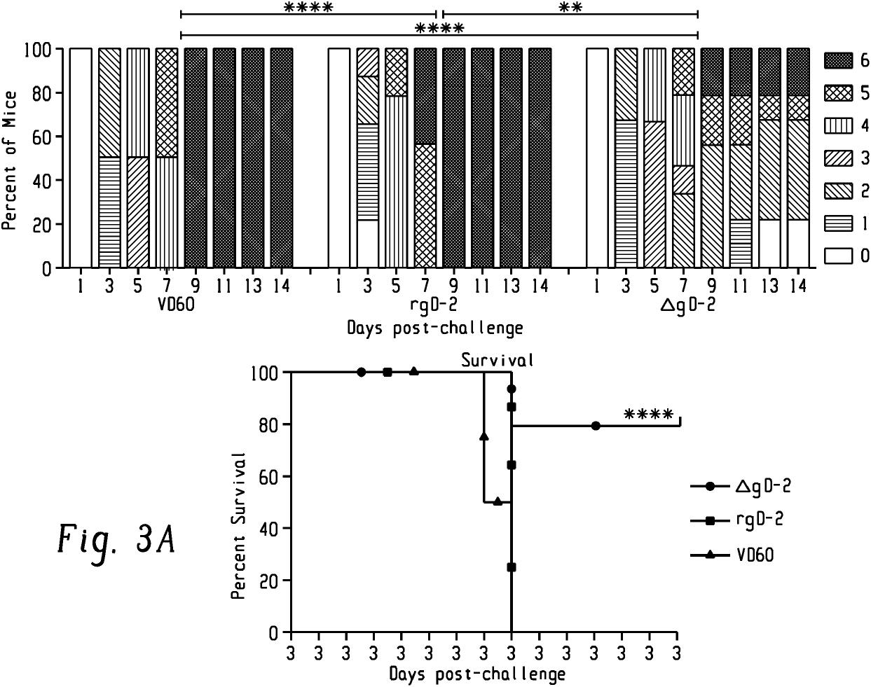

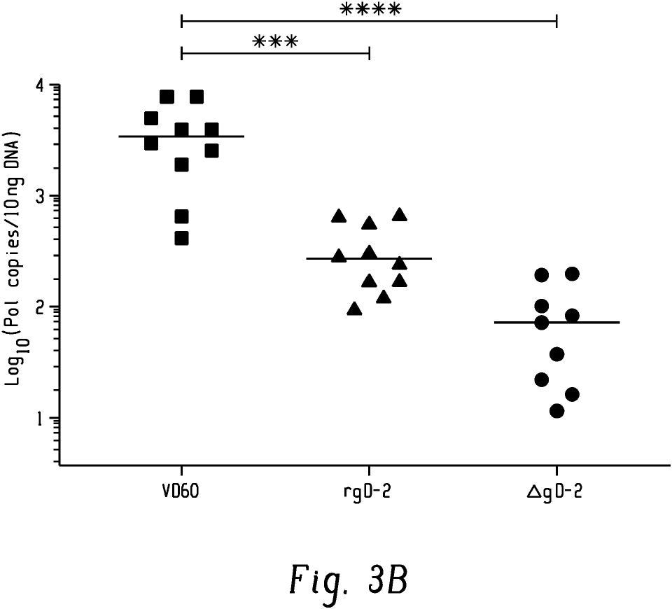

Previous studies demonstrated that immune serum from ΔgD-2 vaccinated mice passively protected naive animals from vaginal or skin challenge (Petro et al, 2015, Elife 4). To assess whether immune serum could also protect against ocular disease, serum from ΔgD-2, rgD-2/Alum-MPL or control-immunized mice was pooled and an equivalent amount of total IgG (750 µg) was administered intraperitoneally one day prior and four days following corneal challenge with 10⁵ pfu of B3x1.1 Although all mice developed signs of disease, the majority (8/10) that received ΔgD-2 immune serum survived, whereas none of the mice that received rgD-2/Alum-MPL or VD60 immune serum survived (FIG. 3A, p< 0.0001). Disease scores were higher and progressed more rapidly in the latter groups. Passive transfer of ΔgD-2 immune serum reduced the quantity of HSV-1 DNA detected in TG compared to VD60 (p< 0.0001). Pooled rgD-2/Alum-MPL immune serum also reduced the amount of latent TG virus compared to VD60 immune serum (p<0.001) despite not providing any survival benefit (FIG. 3B).

### Example 3: Non-Neutralizing Antibodies Protect Against Ocular Disease

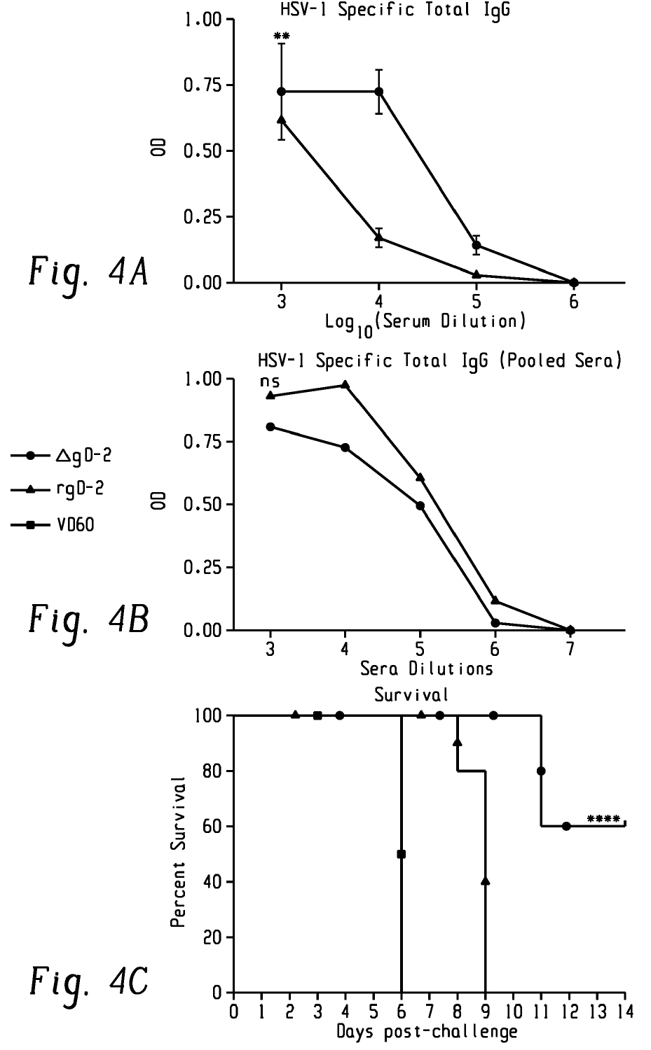

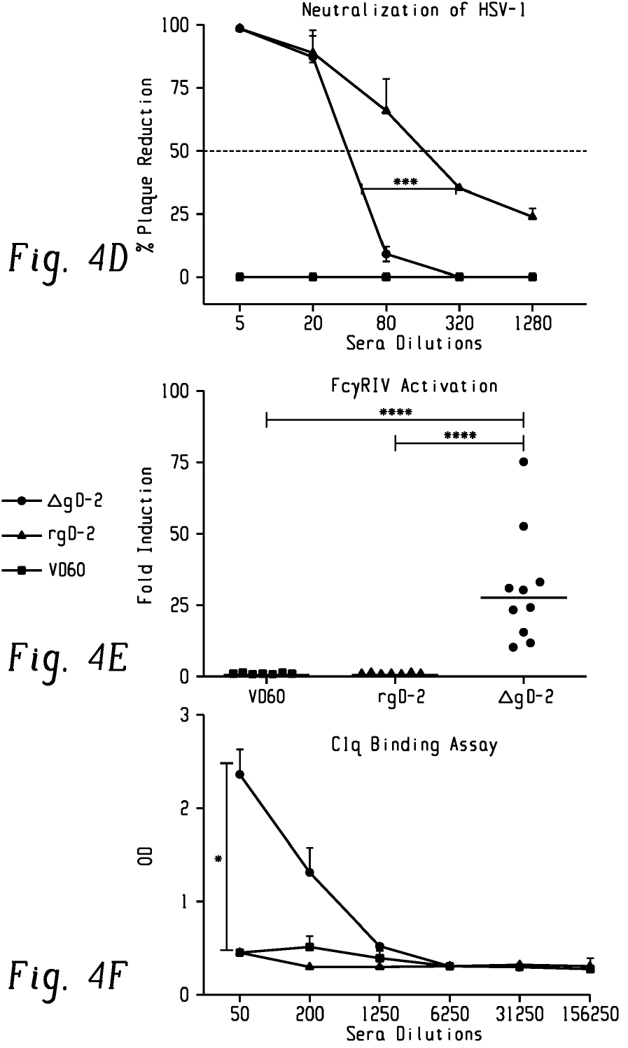

The striking loss in protection following passive compared to active immunization with rgD-2/Alum-MPL (0/10 versus 8/10 survivors, respectively), but not with ΔgD-2 (8/10 and 10/10 survivors) could be attributed to quantitative and/or qualitative differences in the antibody response and/or relative contribution of T cells to immune protection with the two vaccines. ΔgD-2 elicited higher total HSV-1 specific IgG responses compared to rgD-2/Alum-MPL (FIG. 4A). To assess whether this quantitative difference accounted for the lack of passive protection afforded by rgD-2/Alum-MPL, new pools were prepared by combining immune serum from individual mice with similar concentrations of HSV-1 antibody titer (measured by ELISA) and the HSV specific IgG in the new pools compared (FIG. 4B). The passive transfer studies were repeated with these new pools and again showed significantly greater protection with ΔgD-2 compared to rgD-2 immune serum, indicating that the concentration of HSV-specific IgG does not fully explain the differences in protection (FIG. 4C). We then compared the functionality of the Ab responses. Consistent with prior studies (5), rgD-2/Alum-MPL elicited a higher titer of neutralizing Abs (FIG. 4D), whereas the Abs elicited by ΔgD-2 exhibited greater activation of the FcγRIV receptor, a biomarker of ADCC (FIG. 4E). Moreover, the antibodies elicited by ΔgD-2, but not the antibodies elicited by rgD-2/Alum-MPL, also bound Clq (FIG. 4F).

### Example 4: T Cell Depletion Following Active Vaccination with Either the Subunit or Single-Cycle Vaccine Leads to a Reduction in Protection

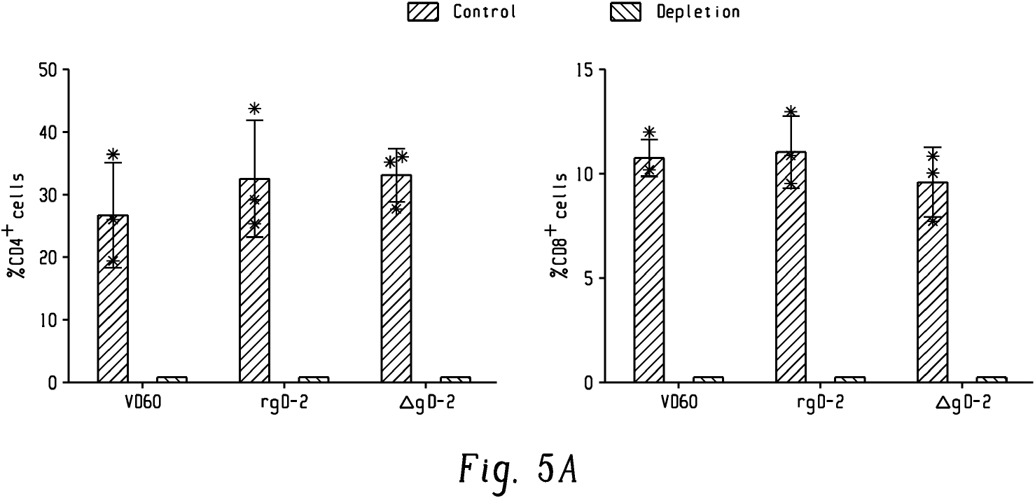

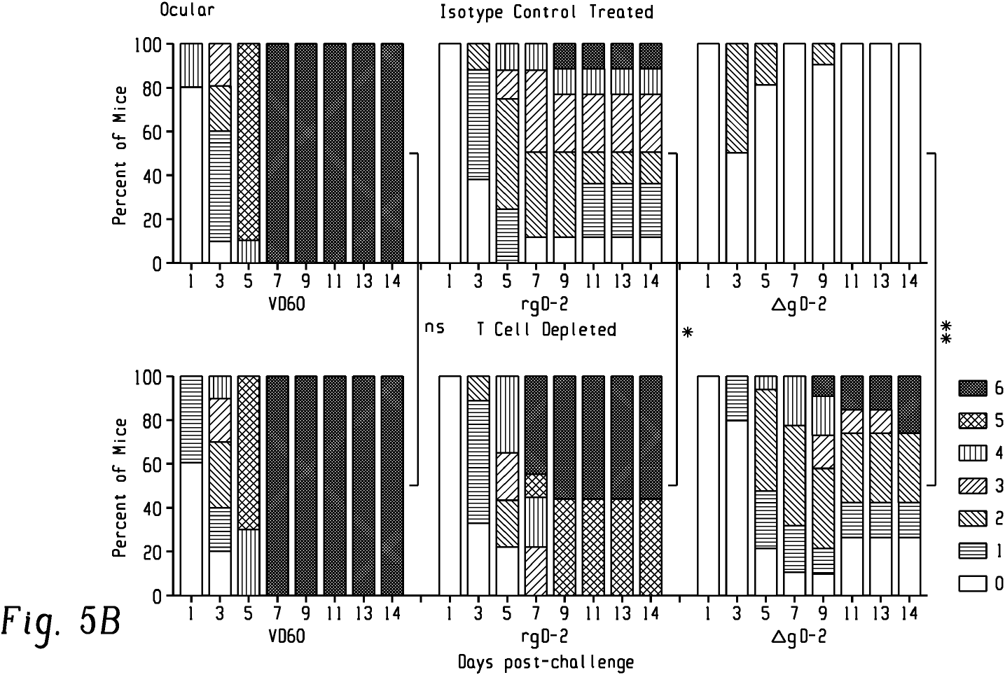

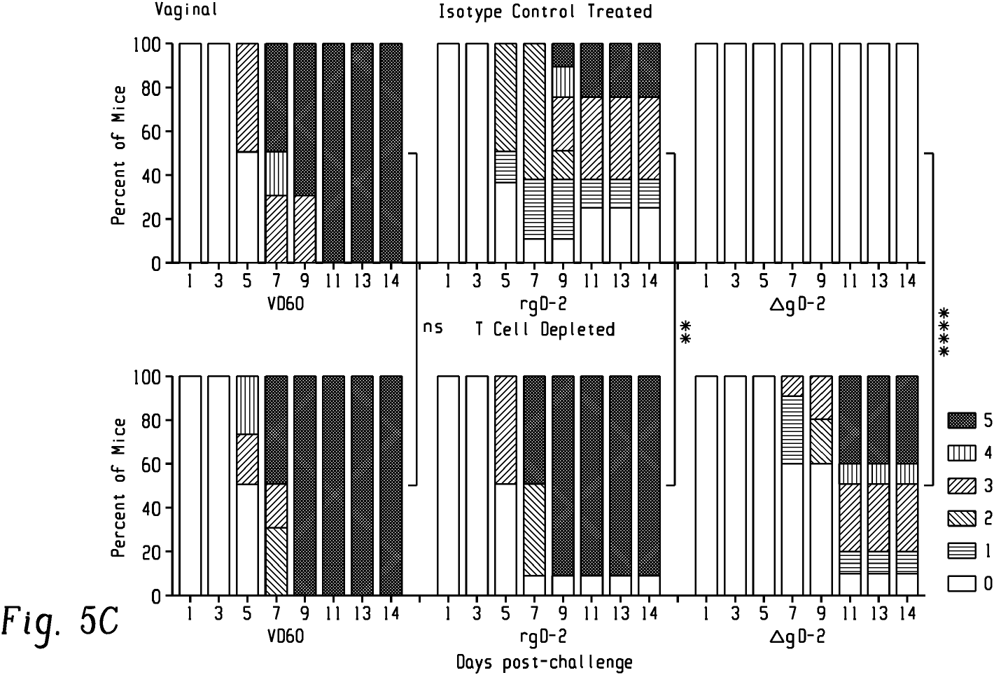

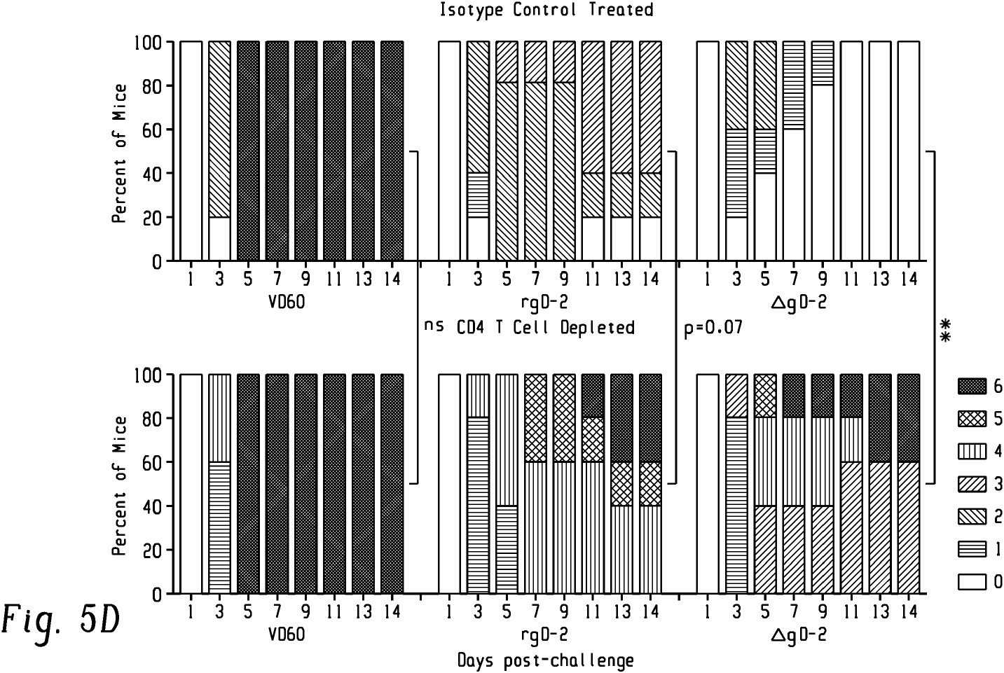

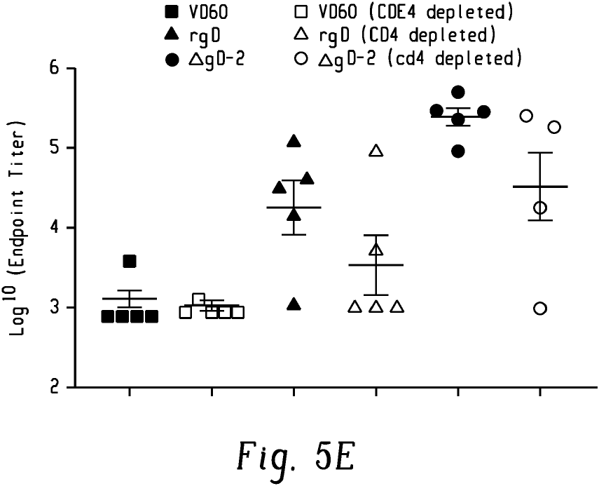

The greater discordance between active and passive protection provided by rgD-2/Alum-MPL compared to ΔgD-2 could also reflect a greater role for T cells in mediating subunit vaccine protection following active immunization. To address this, mice were actively immunized with 2 doses of each vaccine and then T cells were depleted by treating the mice with anti-CD4 and anti-CD8 mAbs 4 days and 2 days prior to ocular or, for comparison, vaginal challenge. Controls received an unrelated isotype-matched Ab. The depleting Abs resulted in a greater than 95% reduction in CD4 and CD8 T cell populations by flow cytometry (FIG. 5A). As expected, ΔgD-2 provided significantly greater protection than rgD-2/Alum-MPL against ocular (FIG. 5B) or vaginal (FIG. 5C) challenge in the isotype-control treated mice. Depletion of T cells just prior to challenge, however, led to a significant reduction in protection for both vaccines. A similar reduction in protection against ocular disease was also observed when only CD4+ T cells were depleted, which trended towards significance for the subunit vaccine (p=0.07) and was significant for ΔgD-2 (p<0.01) (FIG. 5D). Depletion of CD4+ T cells was associated with a modest, but not statistically significant, decrease in HSV-specific Ab titers in the blood, consistent with a role for CD4 T cell in maintaining Ab levels in mice (FIG. 5E) (22, 23).

### Discussion

Following active or passive immunization, the single-cycle ΔgD-2 vaccine strain provided significantly greater protection compared to an adjuvanted gD-2 subunit protein vaccine in a primary ocular murine challenge model using a clinical isolate of HSV-1 at lethal doses. The differences were more striking in the passive transfer studies and persisted after delivering a comparable amount of total HSV-specific IgG, thus providing insights into the relative contribution of functionally distinct Abs in mediating protection. The Abs elicited in response to ΔgD-2 bind and activate the murine FcγRIV, the primary mediator of ADCC, and as shown here for the first time, also bind Clq. The Clq and FcγR-bindings sites on the Fc domain partially overlap and both contribute to immune protection. Engagement of FcγRIV promotes ADCC and antibody-dependent phagocytosis, and binding to Clq activates the complement cascade leading to complement-dependent cytotoxicity. Without being limited by theory, it is believed that in addition to directly activating complement-dependent cytolysis, the ability of the Fc portion of IgG to bind C1q may further promote engagement of the FcγR via bridging, and thereby enhance Fc effector functions. The gD subunit vaccine, in contrast, induces an almost exclusive neutralizing Ab response with little or no FcγRIV activation or Clq binding. The inability of the gD-specific neutralizing Abs to provide as much protection as the non-neutralizing Abs may reflect the ability of HSV to spread from infected to uninfected cell across intercellular bridges thereby escaping neutralization. The Abs also differ with respect to antigenic targets with the subunit vaccine primarily generating Abs to gD (Halford, 2014, Expert Rev Vaccines 13:691-710; Whitbeck et al, 2014, Journal of Virology 88:7786-7795), whereas ΔgD-2 elicits responses targeting multiple viral antigens (Petro et al, 2015, Elife 4). In addition, the Abs may differ in their ability to bind the Fc neonatal receptor and be retained within the cornea.

The difference in protection against lethality following active compared to passive protection for rgD-2/Alum-MPL versus ΔgD-2 could not be explained by a differential role for T cells. Depletion of CD4+ and CD8+T cells or CD4+ cells alone from actively immunized mice prior to challenge led to a reduction in protection for both vaccines. The loss of protection was observed following both ocular and vaginal challenges and was somewhat greater in the ΔgD-2-immunized mice. This could reflect a role for CD4 help in facilitating Ab localization to immune privileged sites, including the eye and the peripheral nervous system. The cornea, peripheral nervous system and brain are protected by a blood-ocular, blood-nerve or blood-brain barrier. A recent study showed that CD4+ T cells contribute to the ability of Abs to access these sites by releasing interferon-y to promote an increase in vascular permeability (30). Without being limited by theory, it is believed that this contributed to the decrease in protection observed when T cells were depleted just prior to challenge. Moreover, other immune cell populations, most notably CD4+ dendritic cells (DCs) may be depleted by anti-CD4 treatment. This could have further interfered with ΔgD-2 vaccine efficacy since DCs, which express FcγRIV, contribute to antibody-mediated cell killing in mice (Vafa et al, 2014, Methods 65:114-26; Davido et al, 2018, J. Virol. 92). We found that depletion of CD1 1c+ cells led to a loss of protection in passive transfer studies with ΔgD-2 immune serum (Burn Aschner and Herold, unpublished).

The ocular model used in these studies differs from the model in which mice are challenged with sublethal doses of laboratory-adapted strains of HSV-1 that cause ~50% or less mortality (Keadle et al, 1997, J Infect Dis 176:331-8; Royer et al, 1050, Journal of immunology 199:1898-1911; van Lint et al, 2007, Virology 368:227-31; Davido et al, 2018, J Virol 92). Potential advantages of the high dose lethal challenge model include its stringency, but a disadvantage is that it does not reflect clinical ocular disease, which is not lethal and more often the result of repeated episodes of viral reactivation. However, the observation that vaccination with ΔgD-2 was associated with rapid clearance of virus, protection against dissemination of virus to the contralateral trigeminal ganglia, and a significant decrease in the latent reservoir, as measured by qPCR in the ipsilateral trigeminal ganglia, provides strong indirect evidence that this different vaccine strategy would prevent or reduce the risk of reactivation. Although we did not attempt to induce reactivation with UV light or other stimulation in the ΔgD-2 vaccinated mice, no signs of ocular disease were clinically observed in any of the vaccinated mice who were monitored for up to 4 weeks post-challenge. Future studies with models of recurrent disease such as rabbit models may provide further insights.

Passive transfer of immune serum from ΔgD-2 vaccinated mice was less effective against ocular disease compared to vaginal or skin disease. A single dose of pooled ΔgD-2 immune serum (containing 750 µg of total IgG) administered one day prior to challenge provided 100% protection against a lethal HSV-2 vaginal or skin challenge (Petro et al, 2015, Elife 4). Complete protection against lethality was also observed in male mice treated with a single dose of ΔgD-2 immune serum; notably in that study, immune serum pooled from rgD-2/Alum-MPL immunized mice provided no protection against lethal skin challenge (Burn et al, 2017, J Infect Dis., doi:10.1093/infdisJjix628). The reduction in efficacy with ΔgD-2 immune serum in the prevention of ocular compared to vaginal or skin disease, even when two doses were administered to address the relatively short half-life (4-8 days) of murine IgG (Mankarious S, et al, 1988, J. Lab. Clin. Med., 112:634-40; Vieira et al, 1988, J. Immunol., 18:313-6), likely reflects accumulation of less Ab in the eye, either because of differences in vascular permeability and/or ability of the FcRn to pump Ab out of the eye (Kim et al, 2009, Mol. Vis., 15:2803-12).

The novelty of the current studies is that non-neutralizing Abs accumulate sufficiently in the immune privileged eye following active or passive immunization to provide even greater protection against lethal ocular disease. These findings may have implications not only for development of strategies to prevent or treat ocular HSV but may be relevant for other pathogens.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A method of treating ocular disease caused by herpes simplex virus-1 (HSV-1) infection, comprising administering to a subject an effective amount of a herpes simplex virus-2 (HSV-2) having a deletion of an HSV-2 glycoprotein D-encoding gene in the genome to treat the ocular disease in the subject, wherein the HSV-2 is phenotypically complemented with an HSV-1 glycoprotein D by propagating the HSV-2 in a complementing cell expressing HSV-1 glycoprotein D.

2. (canceled)

3. The method of claim 1, wherein the HSV-2 is a single cycle virus.

4. The method of claim 1, wherein the ocular disease caused by HSV-1 comprises blepharitis, conjunctivitis, keratitis, retinitis, scleritis, or a combination thereof.

5. The method of claim 1, wherein the HSV-2 is a component of a pharmaceutical formulation.

6. The method of claim 5, wherein the pharmaceutical composition further comprises an adjuvant.

7. The method of claim 1, wherein the subject is receiving or has received an anti-viral drug.

8. The method of claim 7, wherein the anti-viral drug comprises acyclovir.

9. The method of claim 1, wherein the subject is infected with HSV-1.

10. The method of claim 1, wherein the subject is not infected with HSV-1.

11. The method of claim 1, wherein the deletion of the HSV-2 glycoprotein D-encoding gene comprises a deletion of the entire HSV-2 glycoprotein D-encoding gene in the genome of the HSV-2.

12. A method of treating ocular disease caused by herpes simplex virus-1 (HSV-1) infection in a first subject comprising administering to the first subject an effective amount of a product from a second subject to treat the ocular disease in the first subject, wherein the second subject is immunized with a herpes simplex virus-2 (HSV-2) having a deletion of an HSV-2 glycoprotein D-encoding gene in the genome and wherein the HSV-2 is phenotypically complemented with a HSV-1 glycoprotein D by propagating the HSV-2 in a complementing cell expressing HSV-1 glycoprotein D, and wherein the product comprises an antibody, an immune factor, or a combination thereof induced by the HSV-2.

13. The method of claim 12, wherein the ocular disease caused by HSV-1 comprises blepharitis, conjunctivitis, keratitis, retinitis, scleritis, or a combination thereof.

14. The method of claim 12, wherein the method elicits an immune response in the first subject against an HSV-1.

15. The method of claim 12, wherein the product comprises an antibody elicited by immunization of the second subject with the HSV-2 or serum from the second subject.

16. (canceled)

17. (canceled)

18. (canceled)

19. The method of claim 15, wherein the antibody comprises a polyclonal antibody, a monoclonal antibody, or a combination thereof.

20. The method of claim 12, wherein the product is a component of a pharmaceutical formulation.

21. The method of claim 12, wherein the method comprises passive transfer of the product from the second subject to the first subject.

22. A method of treating ocular disease caused by herpes simplex virus-1 (HSV-1) infection in a first subject comprising administering to the first subject an effective amount of an antibody specific for an antigen of the HSV-1 to treat the ocular disease in the first subject, wherein the antibody is obtained by immunizing a second subject with a herpes simplex virus-2 (HSV-2) having a deletion of an HSV-2 glycoprotein D-encoding gene in the genome and wherein the HSV-2 is phenotypically complemented with an HSV-1 glycoprotein D by propagating the HSV-2 in a complementing cell expressing HSV-1 glycoprotein D, and wherein the antibody elicited by immunizing the second subject with the HSV-2 comprises an Fc receptor-activating antibody.

23. The method of claim 15, wherein the product is a component of a pharmaceutical formulation.

24. The method of claim 19, wherein the product is a component of a pharmaceutical formulation.
